(19) United States
(12) Patent Application Publication
(10) Pub. No.: US 2018/0161538 A1
(43) Pub. Date: Jun. 14, 2018

(54) THERMOPLASTIC STRIKE LAYER FOR BONDING MATERIALS

(71) Applicant: St. Jude Medical Puerto Rico LLC, Caguas, PR (US)

(72) Inventors: Brent Ford, Minneapolis, MN (US); Timothy S. Marass, Minneapolis, MN (US); Nicholas Strom, Minneapolis, MN (US); John F. Arnold, Andover, MN (US)

(73) Assignee: St. Jude Medical Puerto Rico LLC, Caguas, PR (US)

(21) Appl. No.: 15/827,949

(22) Filed: Nov. 30, 2017

Related U.S. Application Data
(60) Provisional application No. 62/433,631, filed on Dec. 13, 2016.

(51) Int. Cl. A61M 25/00; B29C 65/02; B32B 27/08; B32B 27/28; B32B 27/34; B32B 27/36; B32B 27/40; B29C 41/00; B29C 41/02

(52) U.S. Cl. CPC A61M 25/001; B29C 65/02; B32B 27/08; B32B 27/281; B29K 2071/00; B32B 27/365; B32B 27/40; B29C 41/003; B29C 41/02; B32B 27/34

(57) ABSTRACT

Apparatuses and methods are disclosed that may be used to bond materials using thermoset and thermoplastic polymer materials arranged with a thermoplastic strike layer on a surface of the material. Apparatuses and methods are also disclosed for manufacturing and configuring catheters and other tubular devices with a bonding surface or layer. These apparatuses and devices may have a strike layer of thermoplastic polymer material that melts or softens upon application of heat and bonds to other polymer materials upon cooling, thus avoiding the use of externally-applied adhesives in the construction of a bond between two surfaces, such as between an inner surface of a tubular catheter and an outer surface of a tubular catheter tip.

### RELATED APPLICATION

This claims the benefit of U.S. Provisional Application No. 62/433,631, filed on 13 Dec. 2016, which is hereby incorporated by reference herein in its entirety.

### TECHNICAL FIELD

The present disclosure generally relates to apparatuses and methods related to bonding materials such as polymer materials to each other using a strike layer of thermoplastic polymer material positioned between the materials, and the disclosure particularly relates to medical devices using the strike layer to bond materials.

### BACKGROUND

Flexible catheters and other tubing are used in many applications and product lines in the field of medical devices. Medical-grade catheters are often used to deliver or drain fluids within a patient, and are used to support and direct the movement of guidewires, diagnostic and surgical instruments, and medications. Different types of catheters are employed depending on the application, including catheters that meet specific requirements for size, length, flexibility, stiffness, and chemical properties. Each type of catheter may be made of different materials and may be made using different manufacturing processes in order to ensure a suitable end-product.

For some catheters, the properties of the distal tip (i.e., the end portion of the catheter that is inserted most deeply into the patient) are critical to the function and safety of the catheter as a whole. For example, an electrode (or other device) may be delivered at the distal tip of the catheter to a treatment zone, treatment may be administered by the electrode, and the electrode and catheter may then be removed. During this procedure, the structure and integrity of the tip are vital to ensure that the electrode stays in the proper orientation relative to the rest of the catheter and so that the electrode does not become disconnected from the catheter or lost within the body. However, electrodes and other devices used at the distal end of a catheter cannot be efficiently and economically manufactured to be integrally formed as part of the rest of the catheter, which is usually formed by an extrusion process. Therefore, distal devices such as an electrode tip must be attached to the distal end of the catheter prior to use of the catheter within the body.

Current methods of attaching devices to the end of a catheter are defect-prone and can be time-consuming. Externally-applied adhesives are often used in the methods that can easily spread into parts of the joint between the catheter and the tip where they occlude flow through the catheter lumen or other parts of the tip (e.g., lateral flow ports through the tip wall). The adhesives also commonly have physical and chemical properties and limitations that are different from the materials used for the catheter and tip, so it is common for the adhesive, once cured, to change the flexibility of the joint area. Added adhesive also undesirably changes the thickness and diameter of the joint area and can take an excessive amount of time to apply and cure. Accordingly, there is a need for improvements to bonding methods used to join parts in medical devices.

### SUMMARY

One aspect of the present disclosure relates to a tube having a bonding surface. The tube may comprise a tubular body having a central lumen, wherein the tubular body may comprise a longitudinal axis. The tube may also have a tubular thickness (with the tubular thickness extending through the tubular body in a radial direction relative to the longitudinal axis), a first thickness layer comprising a thermoset polymer material, and a second thickness layer comprising a thermoplastic polymer material. The thermoplastic polymer material may comprise at least one of polyether block amide, polyurethane, nylon, or polycarbonate.

In some arrangements, the second thickness layer may comprise the thermoset polymer material and the thermoplastic polymer material. The thermoset polymer material may comprise polyimide and the thermoplastic polymer material may comprise polyether block amide. The thermoplastic polymer material may be bondable to the thermoset polymer material upon heating the second thickness layer. The first thickness layer may be positioned radially internal to the second thickness layer. The first thickness layer may be positioned radially external to the second thickness layer. The first and second thickness layers may be bonded to each other. A third thickness layer may also be included, wherein the third thickness layer may be positioned radially between the first and second thickness layers. The third thickness layer may comprise the thermoset polymer material and the thermoplastic polymer material. The thermoset polymer material and the thermoplastic polymer material may be blended in the third thickness layer.

Another aspect of the disclosure relates to a tubular catheter joint comprising a tubular catheter and a catheter tip. The tubular catheter may comprise a first longitudinal axis and a first surface, with the first surface extending circumferentially around the first longitudinal axis. The catheter tip may comprise a second longitudinal axis, a second surface, and a third surface, with the second surface extending circumferentially around the second longitudinal axis, and the third surface extending circumferentially around the second longitudinal axis and being radially spaced from the second surface. The second surface may comprise a thermoset polymer material and the third surface may comprise a thermoplastic polymer material. The thermoplastic polymer material may be bonded to the first surface. The thermoplastic polymer material may comprise at least one of polyether block amide, polyurethane, nylon, or polycarbonate. The tubular catheter and the catheter tip may be arranged in fluid communication with each other.

In some embodiments of the apparatus, the first surface may be positioned radially external to the third surface. The catheter tip may comprise a first end portion comprising the second and third surfaces and a second end portion positioned opposite the first end portion. The second end portion may have a greater outer diameter than the first end portion. The tubular catheter and catheter tip may each have constant outer diameters along the first and second longitudinal axes. The first surface may be positioned radially internal to the third surface.

Yet another aspect of the disclosure relates to a method of bonding surfaces of polymer materials. The method may comprise providing a first bonding surface and a second bonding surface, with the first bonding surface comprising a thermoset polymer material, the second bonding surface comprising the thermoset polymer material and a thermoplastic polymer material, and the thermoplastic polymer material comprising at least one of polyether block amide, polyurethane, nylon, or polycarbonate. The method may also include applying heat to the second bonding surface, bringing the first and second bonding surfaces into contact with each other, and cooling the first and second bonding surfaces, wherein the thermoplastic polymer material is bonded with the first bonding surface.

The first bonding surface may be positioned on a first device and the second bonding surface may be positioned on a second device. The second device may comprise a first layer and a second layer, with the first layer comprising the second bonding surface, the second layer comprising the thermoset polymer material, and the first layer being bonded to the second layer. In some embodiments, the first and second devices are tubular. Applying heat to the second bonding surface may at least partially melt the thermoplastic polymer material at the second bonding surface. The thermoset polymer material may comprise polyimide. The thermoset polymer material and the thermoplastic polymer material may be blended together in the second bonding surface.

Still another aspect of the disclosure relates to a method of forming a tubular catheter. The method may comprise providing a mandrel, coating the mandrel with a first polymer material, with the first polymer material comprising a thermoset polymer and the thermoset polymer forming a tubular shape around the mandrel. The method may also include coating the tubular shape with a second polymer material, with the second polymer material comprising the thermoset polymer and a thermoplastic polymer and the thermoplastic polymer comprising at least one of polyether block amide, polyurethane, nylon, or polycarbonate. The method may also comprise curing the first and second polymer materials and removing the mandrel from the first and second polymer materials.

The thermoset polymer and the thermoplastic polymer may be blended in the second polymer material. The second polymer material may comprise an inner boundary and an outer boundary, wherein the outer boundary may have a higher concentration of the thermoplastic polymer than the inner boundary.

In some arrangements, the method may further comprise providing a second tubular member, with the second tubular member having an inner surface, approximating the inner surface of the second tubular member and the second polymer material, heating the second polymer material, thereby at least partially melting the thermoplastic polymer, and bonding the second polymer material to the inner surface of the second tubular member. The step of approximating the inner surface of the second tubular member and the second polymer material may comprise positioning an attachment mandrel within the first polymer material, and the attachment mandrel may remain within the first polymer material until the second polymer material is bonded to the inner surface of the second tubular member.

The above summary of the present invention is not intended to describe each embodiment or every implementation of the present invention. The Figures and the detailed description that follow more particularly exemplify one or more preferred embodiments.

### BRIEF DESCRIPTION OF THE DRAWINGS

The accompanying drawings and figures illustrate a number of exemplary embodiments and are part of the specification. Together with the present description, these drawings demonstrate and explain various principles of this disclosure. A further understanding of the nature and advantages of the present invention may be realized by reference to the following drawings. In the appended figures, similar components or features may have the same reference label.

FIG. 1 is a partial cross-sectional side view of an interface between a catheter and a catheter tip during manufacturing of a conventional embodiment.

FIG. 2 is another partial cross-sectional side view of an interface between a catheter and a catheter tip according to the conventional embodiment.

FIG. 3 is another partial cross-sectional side view of an interface between a catheter and a catheter tip according to the conventional embodiment.

FIG. 4 is another partial cross-sectional side view of an interface between a catheter and a catheter tip according to the conventional embodiment.

FIG. 5 is a cross-sectional side view of an end of a tube according to an embodiment of the present disclosure.

FIG. 5A is a cross-sectional end view of the tube of FIG. 5.

FIG. 6 is a partial cross-sectional side view of an interface between a catheter and a catheter tip during manufacturing according to an embodiment of the present disclosure.

FIG. 7 is another cross-sectional side view of an interface between a catheter and a catheter tip according to an embodiment of the present disclosure.

FIG. 8 is another cross-sectional side view of an interface between a catheter and a catheter tip according to an embodiment of the present disclosure.

FIG. 9 is a cross-sectional side view of a tube according to another embodiment of the present disclosure.

FIG. 9A is a cross-sectional end view of the tube of FIG. 9.

FIG. 10 is a cross-sectional side view of a tube according to another embodiment of the present disclosure.

FIG. 10A is a cross-sectional end view of the tube of FIG. 10.

FIG. 11 shows a flow diagram illustrating an example method of manufacturing a tubular catheter according to an embodiment of the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The present disclosure generally relates to apparatuses and methods related to bonding materials used in medical devices such as catheters and other intravascular surgical equipment. Materials in these medical devices are carefully selected for their chemical and mechanical properties, such as heat resistance, flexibility, chemical and biological reactivity and stability, and tensile strength. Reactivity and heat resistance are some of the most important characteristics in medical devices since the tools are used in the human body and therefore should have low reactivity and high heat resistance to prevent internal degradation or failure. These devices are therefore commonly constructed with relatively inert and stable polymer materials such as polyimide. However, the low reactivity of these materials can make them difficult to bond with other materials, especially with other portions of a medical device that use the same material. Aspects of the present disclosure relate to ways for inert polymer materials to be bonded to other inert polymer materials using a strike layer.

As used herein, a “strike layer” refers to a layer of material on a surface or part of a surface. The strike layer is intended to work as an intermediary or connecting bridge between two components. The strike layer may be used to join two materials that otherwise would not be able to directly bond to each other. In some embodiments, the strike layer may be a coating or layer added to the outside of a component, but in other embodiments, the strike layer may be part of the thickness of the component itself. Thus, the strike layer may refer to a portion of the thickness of a surface that is intended to work as an intermediary or connecting bridge between the surface and another surface.

In one aspect of the disclosure, apparatuses and methods are set forth for attaching two device components to each other. In one embodiment, the first component comprises a first surface and the second component comprises a second surface. The first surface may comprise an inert thermoset polymer such as, for example, polyimide, polycarbonate, high density polyethylene, or the like. The second surface may comprise an inert thermoset polymer (which may be the same as the polymer used in the first surface) stably infused and blended with a thermoplastic polymer such as, for example, polyether block amide (also known as PEBAX®) or thermoplastic polyurethane, nylon, or polycarbonate.

In order to attach the surfaces to each other, heat may be applied to the second surface in a degree and manner sufficient to induce melting or softening of the thermoplastic polymer infused in the second surface. Softening or melting the thermoplastic polymer material may cause the thermoplastic polymer material to transition from a solid state to a flowable or gel-like state. Typically, this heat is not sufficient to induce melting or softening of the thermoset polymer(s) of the first or second surfaces. Thus, the thermoplastic polymer of the second surface may be softened or begin to melt, but thermoset polymer of the second surface retains the general shape of at least a portion of the second component (e.g., a core portion of the second component). The first surface is brought into contact with the second surface while the second surface is softened or melted (whether during or after heat application), and the softened or melted thermoplastic polymer bonds with and adheres itself to the first surface. As the joint between the first and second components cools, the first and second surfaces are bonded together by the thermoplastic polymer of the second component when it re-solidifies. As used herein, a “joint” may be a connection between two components of a device, such as, for example, a connection between the ends or end portions of two separate tubular catheters or a connection between the ends or end portions of a catheter and a catheter tip.

Using this method, the joint between the two device components may be secured to create a tubular catheter apparatus without applying an external adhesive, epoxy, or resin. Therefore, there is less risk of adhesive flowing out of the intended joint area or connection area between the device components. This may be advantageous with catheter devices in particular since their small inner lumens and side openings can be easily clogged or plugged by adhesive that flows into and hardens within the lumens or openings while the joint is assembled. Using methods of the present disclosure, the outer surface of one catheter tube portion can be joined to the inner surface of another catheter tube portion with the thermoplastic material more localized and precisely positioned by the strike layer to hold the joint together without blocking an internal lumen. Adhesives, epoxies, and resins also take significantly longer to apply and cure than the time needed for a joint to be briefly heated and cooled using methods of the present disclosure. Furthermore, even if the applied adhesives do not block a lumen in conventional devices, they still usually alter the external dimensions, rigidity, and other chemical or mechanical properties of the catheter in detrimental ways. Using a strike layer preserves the properties of the catheter such that the catheter only takes on properties of the component parts rather than properties of the adhesive or other material used to bond those parts together.

Another aspect of the present disclosure relates to methods of constructing a device with a strike layer. A mandrel may be coated with a first polymer material such as a thermoset polymer. The first polymer material may therefore form a tubular-shaped coating around the mandrel as it cures. The tubular shape may then be coated with a second polymer material comprising a blended composition of the first polymer material and a second polymer material, wherein the second polymer material comprises a thermoplastic polymer such as polyether block amide, polyurethane, nylon, or polycarbonate. The mandrel may be removed from the tubular shape, leaving behind an internal lumen. The outer coating may form a strike layer for the tubular device. In some embodiments, many layers of polymer materials are applied to the exterior of the first polymer material. While the inner-most layer of the tube may comprise a pure or essentially pure thermoset polymer, the composition of the tube may gradually, progressively, and/or continuously change as each layer of polymer material is applied. When the tube is completed, the inner-most layers of the tube may have a relatively low concentration of thermoplastic material, and the outer-most layers of the tube may have a relatively high concentration of thermoplastic material, with a gradual and/or continuous material transition in between.

When the device thickness has a gradual material transition, heat applied to the tube softens and/or melts the outer layers of the tube, but inner layers are less likely to move relative to the outer layers. Thus, the device is more stable during joint assembly when the outer strike layer(s) are softened or melt relative to the inner layer(s). In some embodiments, the gradual material transition is achieved using discrete layers that each have discrete and different amounts of intermixture of the thermoset and thermoplastic polymer materials, with a clear separation of different materials between each layer. For example, a catheter may comprise three layers: a first layer having a nearly pure thermoset polymer composition, a second layer external to the first layer that comprises the thermoset polymer blended with a thermoplastic polymer at a first concentration, and a third layer external to the second layer that comprises the thermoset polymer with the thermoplastic polymer at a second concentration that is higher than the first concentration. Other embodiments may comprise more than three layers. In still other embodiments, the number of layers within the thickness of the catheter is great enough, and the layers thin enough, that the layers blend together continuously and without a clear separation or difference between adjacent layers.

Embodiments of the present disclosure may be used in the construction and assembly of catheters and other tubular members, but the scope of the present disclosure is not limited to these devices. Principles of the present disclosure may be applied to other devices, including, for example, other medical devices, wherein a strike layer may be used to bond two surfaces to each other that are not necessarily tubular or cylindrical in shape. For instance, two flat surfaces may be bonded together using the teachings of the present disclosure.

Embodiments of the present disclosure may be used in various different types of devices and with different component parts of various devices. For example, catheter tubes may be manufactured by bonding a first tube portion to a second tube portion that uses a strike layer of thermoplastic polymer-infused material. The processes and methods may be used to connect a tip to the distal end of a catheter and/or may be used to connect a hub or other instrument to a proximal end thereof. The present description provides examples, and is not limiting of the scope, applicability, or configuration set forth in the claims. Thus, it will be understood that changes may be made in the function and arrangement of elements discussed without departing from the spirit and scope of the disclosure, and various embodiments may omit, substitute, or add other procedures or components as appropriate. For instance, the methods described may be performed in an order different from that described, and various steps may be added, omitted, or combined. Also, features described with respect to certain embodiments may be combined in other embodiments.

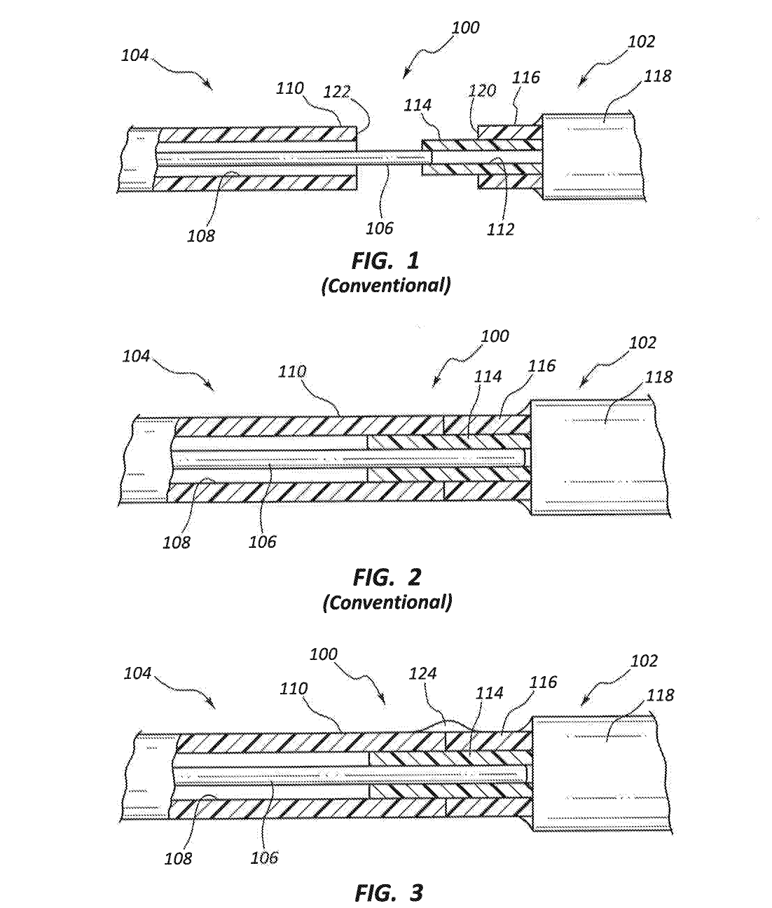

Referring now to the figures in detail, FIGS. 1-4 illustrate section views of steps of a conventional method of constructing a catheter joint 100 by attaching a tip 102 to a catheter 104. Catheter joints 100 may be commonly assembled using this method.

A mandrel 106 may be positioned within the internal lumen 108 of the catheter 104 and advanced through the catheter 104 to its distal end 110. The mandrel 106 may be advanced into a lumen 112 in the tip 102. See FIG. 1. The tip 102 may comprise an inner tube 114 that is bonded to an outer tube 116 and a tip feature or instrument 118. The tip 102 may be moved relative to the catheter 104 so that the inner tube 114 is advanced into the internal lumen 108 of the catheter 104. See FIG. 2. At this point, the proximal end surface 120 of the outer tube 116 is approximated or placed in contact with a distal end surface 122 of the catheter 104. See FIGS. 1-2. As shown in FIG. 2, the outer diameters of the catheter 104 and the outer tube 116 are about equal to each other where the distal end surface 122 and the proximal end surface 120 come together.

While the distal end surface 122 and proximal end surface 120 are approximated, a small amount of a liquid adhesive material (i.e., an adhesive tack 124) is added to at least a portion of the joint area, as shown in FIG. 3. This adhesive tack 124 may hold the positions of the end surfaces 120, 122 relative to each other while the rest of the manufacturing process takes place. The adhesive material typically takes some time to cure, such as about 30 seconds. Upon curing, the manufacturer may inspect the joint, make sure the constituent parts are properly oriented and positioned, and prepare the assembly for application of additional adhesive without the end surfaces 120, 122 coming apart. The manufacturer also commonly makes sure that the mandrel 106 can slide relative to the joint and examines the interior of the lumen 108. The low-viscosity adhesive material can undesirably flow into the lumen 108 and bond to the mandrel 106 or restrict flow in the lumen 108, in which case the joint assembly must be scrapped.

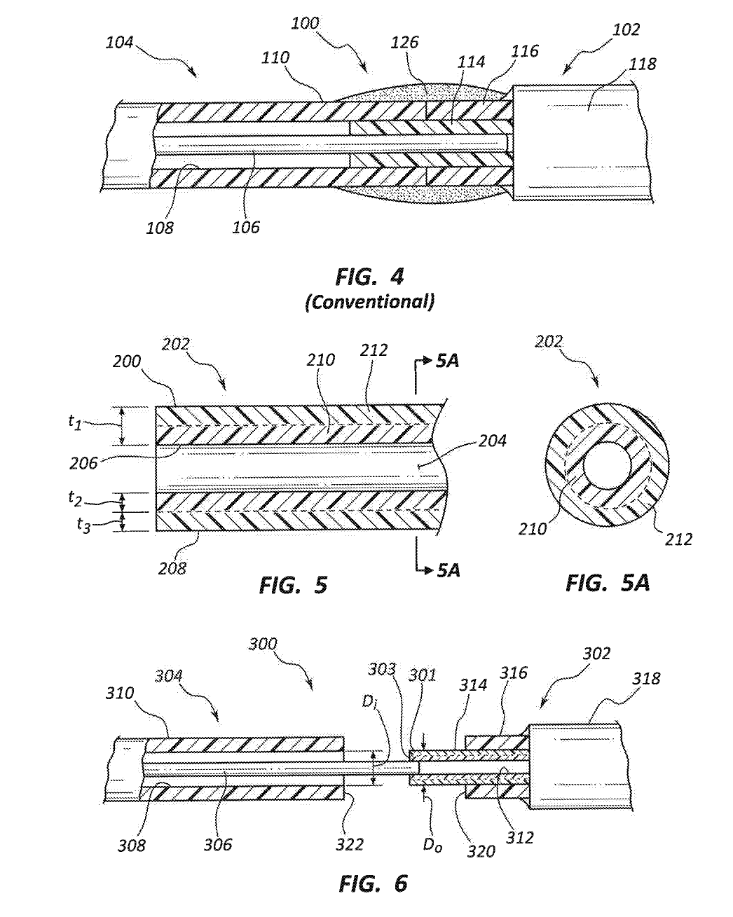

After checking the joint, additional liquid adhesive material is applied to the exterior of the joint. FIG. 4 shows how additional adhesive 126 is applied in a manner extending around the circumference and sealing the interface between the catheter 104 and the tip 102. The additional adhesive 126 then cures over about 30 seconds. The mandrel 106 and lumen 108 are again inspected to make sure adhesive material has not flowed into the lumen 108 or bonded to the mandrel 106. The joint must also be inspected to ensure that enough additional adhesive 126 has been applied to achieve a complete seal. A third layer of adhesive may also be applied to seal any portions of the joint that were not previously sealed and to strengthen the joint. This additional layer also takes about another 30 seconds to cure.

The process shown in FIGS. 1-4 requires a relatively large amount of waiting on behalf of the person or machine manufacturing the joint due to the time needed to cure each application of the adhesive material. Thus, the process is undesirably slow. The failure rate is also undesirably high due to runout of the adhesive liquid. Additionally, even when the joint is successfully assembled, the physical properties of the catheter 104, inner tube 114, and outer tube 116 are affected by the application of the adhesive. For example, the adhesive affects the flexibility and diameter of the area around the joint. By comparison, embodiments of the present disclosure may help reduce the rate of failure, provide better control of the bonding process, and facilitate giving designers more options in the design and function of the joint.

FIG. 5 shows a partial section view of a proximal tip 200 of a tube 202 according to an embodiment of the present disclosure. FIG. 5A shows a sectional end view of the tube 202. In this embodiment, the proximal tip 200 comprises an inner lumen 204, an inner surface 206, an outer surface 208, and a total thickness t₁ extending between the inner surface 206 and the outer surface 208. The tube 202 may comprise an inner layer 210 and an outer layer 212 within the total thickness t₁, wherein the outer layer 212 is radially external to the inner layer 210. The outer layer 212 may extend along the entire length of the inner layer 210 and/or along the entire length of the tube 202.

The inner layer 210 may comprise a first polymer material. The first polymer material may be an inert thermoset polymer material such as, for example, polyimide, polycarbonate, high density polyethylene (HDPE), or other polymers with similar rigidity and reactivity. Thus, the first polymer material may be configured to stably keep the shape of the inner layer 210, particularly the inner surface 206, while permitting passage of fluids, instruments, and medications and other chemicals through the lumen 204. The inner layer 210 may have a surface treatment on its surface that comes into contact with the outer layer 212. The surface treatment may be an etching (e.g., by plasma, chemical, or mechanical etching) to improve the receptiveness of the inner layer 210 to thermally reflowed material of the outer layer 212.

The outer layer 212 may comprise a second polymer material. The second polymer material may comprise a thermoplastic polymer material comprising, for example, at least one of polyether block amide (i.e., PEBAX®), polyurethane, nylon, thermoplastic polycarbonate, and other comparable thermoplastic polymer materials. If the second polymer material comprises polycarbonate and the first polymer material comprises polycarbonate, the first and second polymer materials may each comprise a different kind of polycarbonate, such as the second polymer material comprising a thermoplastic polycarbonate. Typically, the second polymer material may comprise a polymer material that has a melting point less than the melting point of the thermoset material used in the first polymer material. In this manner, when heat is applied to the tube 202, the second polymer material in the outer layer 212 may begin to melt before the inner layer 210 begins to melt. Thus, although the outer layer 212 begins to melt and soften when heat is applied, the inner layer 210 retains its shape properties while heat is applied.

In some embodiments, the outer layer 212 may comprise a blended or mixed composition of the second polymer material and the first polymer material. For example, the outer layer 212 may comprise a blended mixture of polyimide and polyether block amide. The first polymer material of the outer layer 212 may strongly bond with the first polymer material of the inner layer 210, even when heat is applied to the tube 202 and the second polymer material begins to soften or melt. Thus, the presence of the first polymer material in the outer layer 212 may help prevent the outer layer 212 from separating, delaminating, or moving relative to the inner layer 210 when heat is applied to the tube 202. The first polymer material in the outer layer 212 may also help provide rigidity and stability to the outer layer 212 so that as the second polymer material softens, the outer layer 212 still retains its general shape properties as the second polymer material becomes gel-like or flowable.

In comparison to conventional tubes, the tube 202 does not use an additional externally-applied adhesive material to bond the proximal tip 200 to a catheter. The tube 202 may be bonded to an internal surface of a catheter without needing to apply any quantity of an external adhesive. The outer layer 212 may be used as a strike layer that is configured to become at least partially tacky and/or flowable when heat is applied, at which point it is configured to strongly bond to an internal surface as the outer layer 212 cools. The outer layer 212 may be bonded to the inner layer 210 due to the inner and outer layers 210, 212 both having at least a portion of their polymer materials in common. The bond may also be fluid-tight to contain fluid flow through the joint.

The inner layer 210 may have a first thickness t₂, and the outer layer 212 may have a second thickness t₃. The second thickness t₃ may potentially be about 0.0002 inches. The second thickness t₃ may also be made within a range of about 5 percent of the first thickness t₂ to about 200 percent of the first thickness t₂. Various thicknesses may cause different targeted amounts of softening or melting when predetermined amounts of heat are applied.

Another aspect of the present disclosure relates to methods of bonding surfaces of polymer materials to create a tubular catheter joint. FIGS. 6-8 illustrate steps in one embodiment of these methods. A catheter joint 300 may be assembled by placing a mandrel 306 within a lumen 308 of a catheter 304 and positioning the mandrel 306 at a distal end 310 of the catheter 304. The mandrel 306 may be advanced into a lumen 312 within a tip 302. The lumen 312 may be within an inner tube 314 that is bonded to an outer tube 316 on the tip 302. The tip 302 may also comprise a tip feature or instrument 318, such as, for example, an electrode, probe, nozzle, guide, expandable member or balloon, or other device.

The inner tube 314 may comprise at least one outer layer 301 and at least one inner layer 303. The inner layer 303 may comprise a first polymer material, and the outer layer 301 may comprise a second polymer material. The first polymer material of the inner layer 303 may comprise the same materials described in connection with the inner layer 210 of tube 202, and the second polymer material of the outer layer 301 may comprise the same materials described in connection with the outer layer 212 of tube 202. Thus, the outer layer 301 may comprise a strike layer that at least partially comprises a thermoplastic polymer material, and the thermoplastic polymer material may be configured to be bonded with the material used to construct the inner surface of the distal end 310 of the catheter 304.

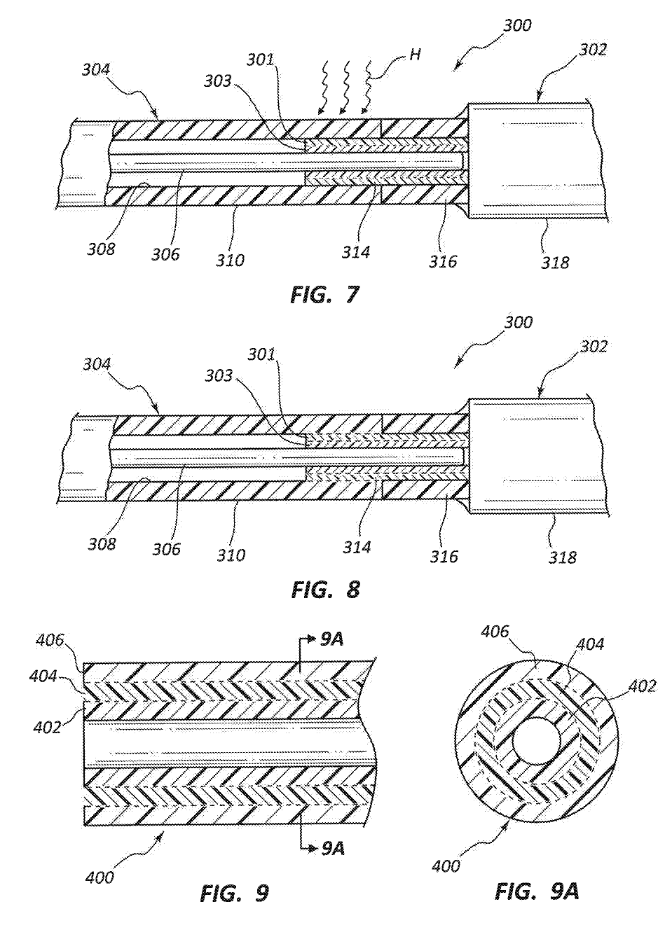

The inner tube 314 may have an outer diameter Dₒ that is substantially equal to the inner diameter Dᵢ of the catheter 304. Thus, the inner tube 314 may be advanced along the mandrel 306 until the outer diameter Dₒ is positioned within the inner diameter Dᵢ of the catheter 304 and within the lumen 308, as shown in FIG. 7. A distal-facing end surface 322 of the catheter 304 may be brought into contact with a proximal-facing end surface 320 of the outer tube 316. See FIGS. 6-7. In the position of FIG. 7, the outer layer 301 of the inner tube 314 is brought close to or into contact with the inward-facing wall of the lumen 308.

Heat H may be applied to warm up the outer layer 301 where the outer layer 301 is near or contacting the lumen 308 of the catheter 304. The heat H softens, melts, liquefies, or otherwise transitions at least some of the thermoplastic material in the outer layer 301 into a sticky solid, viscous liquid, or gel-like state. In that state, the outer layer 301 is attachable to the inner surface of the lumen 308 of the catheter 304. While the outer layer remains softened, the mandrel 306 may remain in place so that the tip 302 and catheter 304 remain in position relative to each other until the outer layer 301 cools and solidifies while attached to the inner surface of the lumen 308. Applied heat H may be cut off from the catheter joint 300 the outer layer 301 has sufficiently bonded with the catheter 304, and the joint 300 may be allowed to cool in place. Upon cooling, the inner tube 314 will be securely bonded to the catheter 304 and to the outer tube 316, as shown in FIG. 8. The mandrel 306 can then be removed from the joint 300 while the tip 302 remains bonded to the catheter 304.

In some embodiments, the heat may be applied directly to the outer layer 301 of the inner tube 314 prior to insertion of the inner tube 314 into the catheter 304. For example, the heat may be applied to the outer layer 301 while it is radially exposed in the position shown in FIG. 6. Depending on the thermal properties of the outer layer 301 and catheter 304, heat may only need to be applied for a very short time, such as about 3-5 seconds, for the outer layer 301 to be softened or melted enough to bond to the catheter 304. Accordingly, these methods of bonding the joint 300 may be significantly faster to complete than conventional methods.

Additionally, the outer layer 301 may not have a viscosity as low as adhesive materials used in conventional methods, so there is a lower likelihood that fluid material will leak into the lumen 308 from the outer layer 301 and either bond to the mandrel 306 or block the lumen 308 in some way. Thus, these methods of bonding the joint 300 may be more reliable and less prone to error than conventional methods.

Furthermore, the present methods do not require the manufacturer to measure and apply adhesive to the joint 300. Instead, the strike layer (i.e., outer layer 301) comes prepared for bonding, and there is less potential for over-application of adhesive. This characteristic also helps make the joint 300 have more consistent properties, since conventional methods may have different stiffness or other properties depending on how much adhesive was applied at the time of manufacture.

Finally, the present methods may allow the features of the joint 300 to be dictated by the properties of the materials used in the catheter 304 and the inner and outer tubes 314, 316 rather than being influenced by the properties of the adhesive used. Thus, the present methods allow a type of control over the properties of the joint 300 that is not available using conventional manufacturing techniques.

FIGS. 9-9A shows an additional embodiment of a catheter 400 according to the present disclosure. FIG. 9 shows a cross-sectional side view, and FIG. 9A shows a cross-sectional end view. Catheter 400 may comprise a plurality of layers, including at least an inner layer 402, an intermediate layer 404, and an outer layer 406. The inner layer 402 may comprise a first polymer material, the intermediate layer 404 may comprise a second polymer material, and the outer layer 406 may comprise a third polymer material. The first polymer material of the inner layer 402 may comprise a thermoset polymer material such as the polymer materials described herein in connection with the inner layer 210 of tube 202. The third polymer material of the outer layer 406 may comprise a blended composition of the thermoset polymer material of the first polymer material of inner layer 402 and a thermoplastic polymer material such as the thermoplastic polymer materials described herein in connection with the outer layer 212 of tube 202. The second polymer material of the intermediate layer 404 may comprise a blended composition of the polymer materials used in the inner layer 402 and the outer layer 406. The intermediate layer 404 may comprise a higher concentration of the thermoset polymer material relative to the thermoplastic polymer material when compared to the third polymer material of the outer layer 406. Thus, the inner layer 402 may entirely comprise the thermoset polymer material, the intermediate layer 404 may comprise a first blended composition of the thermoset polymer material and a thermoplastic polymer material, and the outer layer 406 may comprise a second blended composition of the thermoset polymer material and the thermoplastic polymer material. The first blended composition may comprise a higher concentration of thermoset polymer material than the second blended composition.

In this manner, the outer layer 406 may have higher heat absorption characteristics (i.e., will soften or melt more readily) than the intermediate layer 404, and the intermediate layer 404 may have higher heat absorption characteristics than the inner layer 402. The layered transition between the layers 402, 404, 406 may help prevent individual layers from delaminating or sliding relative to each other when heat is applied and the thermoplastic polymer material is softened since the thermoset polymer material will remain strongly bonded to other thermoset polymer material in other layers. Using the intermediate layer 404 may therefore help hold together the inner layer 402 and the outer layer 406. The intermediate layer 404 may also help preserve the outer dimensions of the outer layer 406 when heat is applied since a lower proportion of the overall thickness of the catheter 400 becomes softened and melts. When heat is applied to the catheter 400, the outer layer 406 may soften to a first hardness or viscosity and the intermediate layer 404 may soften to a second hardness or viscosity, wherein the first hardness or viscosity is less than the second hardness or viscosity.

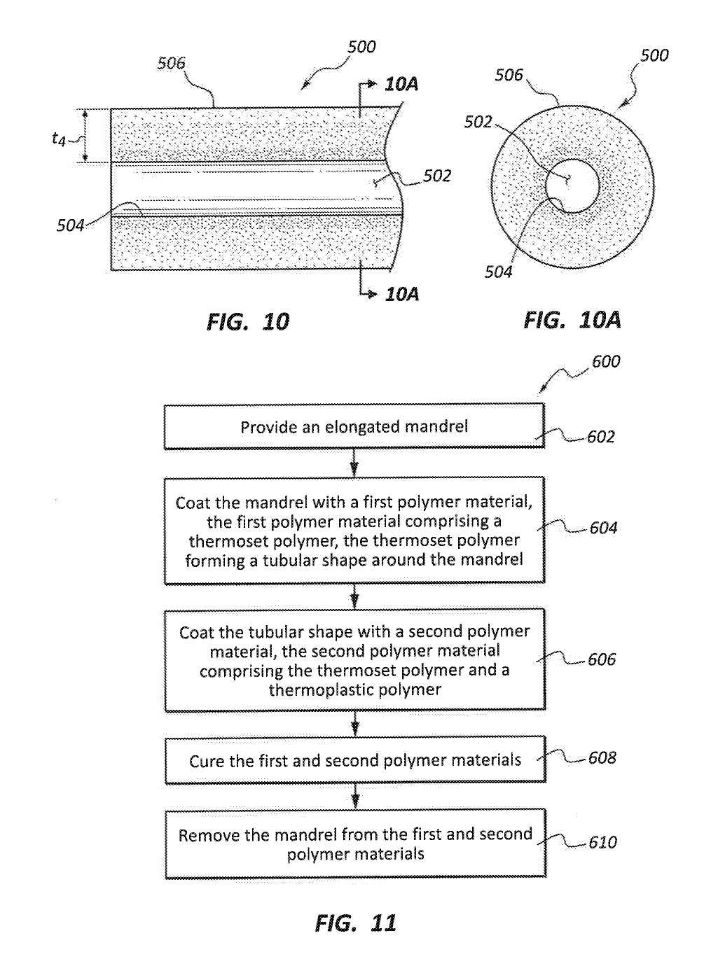

FIGS. 10-10A show yet another embodiment of a catheter 500 according to the present disclosure. FIG. 10 is a cross-sectional side view, and FIG. 10A is a cross-sectional end view. The catheter 500 may be a tubular member comprising an inner lumen 502 having an inner surface 504, an outer surface 506, and a thickness t₄ extending from the inner surface 504 to the outer surface 506. At the inner surface 504 of the catheter 500, the material in the thickness t₄ may comprise substantially entirely a thermoset polymer material such as the thermoset polymer materials described above in connection with the inner layer 210 of tube 202 and the inner layer 402 of catheter 400. Moving radially outward from the inner surface 504, the material in the thickness t₄ may gradually transition from an entirely thermoset polymer material to a blended mixture of the thermoset polymer material and a thermoplastic polymer material, such as, for example, the thermoplastic polymer materials described in connection with other embodiments herein (e.g., the thermoplastic polymer materials in the outer layer 212, intermediate layer 404, and outer layer 406). At the outer surface 506, the composition of the catheter 500 may substantially entirely consist of the thermoplastic polymer material. There is a smooth, continuous transition of material composition from the inner surface 504, to the middle of the thickness t₄ (where there are various degrees of blended materials), to the outer surface 506.

Although a plurality of layers of polymer material may be used to manufacture the catheter 500, the gradual transition across the thickness t₄ of the catheter 500 may lack clear and distinct transitions between layers or a plurality of layers next to each other that share the same material concentration/composition. Thus, unlike the embodiment of FIG. 9, there may be no discrete and distinct layers that each have a specified concentration of polymer materials in the thickness t₄.

In this fashion, the catheter 500 may have a minimized likelihood of layer shearing or delamination since the bonding of thermoset and thermoplastic polymer materials may progressively increase in strength between any given portion of the thickness and a portion of the thickness that is immediately interior to the given portion (aside from the inner surface 504 of the catheter, which does not have any portion interior to it). Shear forces applied to the thickness (e.g., longitudinally-directed forces that would cause delamination or relative longitudinal sliding of layers in the embodiments of FIGS. 5 and 9) do not have distinct layer bodies to act upon wherein the shear forces are concentrated at transitions between the layers. Accordingly, the catheter 500 may be more resistant to shear tearing.

Additionally, because the catheter 500 does not have distinct layers having high concentrations of thermoplastic polymer material, when heat is applied to the catheter 500, there is a lower chance that the outer portions of the thickness t₄ of the tube will melt and flow away from the inner layers of the tube. Instead, heat causes the outer layers to soften and melt, but the gradual transition between the outer surface 506 and inner surface 504 limits the amount of flow since the gradual transition of materials causes the thickness t₄ to have gradually increasing resistance to melting or flowing in the inward radial direction. Thus, the amount of the thickness t₄ melted when heat is applied (or the overall temperature of the catheter 500 is increased) is a continuous function of the amount of heat applied (or the temperature reached). A small amount of heat applied (or a first temperature reached) may melt a small amount of the outer part of the thickness t₄, and a larger amount of heat (or a higher temperature reached) may melt a larger amount of the outer part of the thickness t₄.

By contrast, embodiments with discrete layers may have the same amount of thickness melted at various temperatures or amounts of heat applied. For example, substantially the entire outer layer 212 may melt at a first temperature, and increasing the temperature would not cause it to melt any more of the tube 202 unless the melting temperature of the inner layer 210 were also reached. Accordingly, embodiments with distinct layers may be designed with a predetermined amount of thickness melt for certain applications at certain temperatures, and embodiments with gradual transitions (i.e., without distinct layers) may be designed with an amount of thickness melt based on a function of the temperature. In applications where more melt is needed, the gradually-transitioning catheter 500 may be heated to higher temperatures that melt inner portions of the thickness t₄ that have a higher concentration of thermoset polymer material.

Another aspect of the present disclosure relates to a method for manufacturing a tubular catheter. FIG. 11 shows a flow diagram corresponding with an embodiment of a method for manufacturing the catheter. The method 600 may comprise block 602, which indicates providing a mandrel. The mandrel may be an elongated mandrel similar to the other mandrels (e.g., 306) described and disclosed herein. In some embodiments, the mandrel may comprise a smooth outer surface and an elongated, generally cylindrical shape. In other embodiments, the mandrel may have other smooth outer shape profiles, such as, for example, oval, diamond, ellipse, or other mandrel shapes used for making tubular catheters.

The method 600 may also comprise block 604, wherein the mandrel is coated with a first polymer material. The first polymer material may comprise a thermoset polymer. The thermoset polymer may form a tubular-shaped coating around the outer surface of the mandrel. The tubular shape may comprise a circular inner lumen and outer surface profile, but may alternatively comprise other internal lumen shapes and outer shape profiles such as, for example, an oval, diamond, ellipse, or other tubular catheter shapes. The thermoset polymer of the first polymer material may comprise any of the other thermoset polymer materials described elsewhere herein.

The coating of the first polymer material applied in block 604 may be thin, such as, for example, about 0.0002 inches in thickness. In some embodiments, one layer is applied, and in other embodiments, a plurality of layers of the first polymer material are applied. The plurality of layers may be applied sequentially and in a manner that builds up the thickness of the coating of the first polymer material on the mandrel, such as by allowing initial layers of the polymer material to at least partially cure before applying additional polymer material to the exterior of the initial layers. The coating may form a tubular shape around the mandrel that has substantially equal radial thickness external to the mandrel.

The method 600 may also comprise coating the tubular shape of the first polymer material with a second polymer material, as indicated in block 606. The second polymer material may be evenly applied external to the first polymer material. The second polymer material may comprise at least the thermoset polymer used in the first polymer material and a thermoplastic polymer. The thermoset polymer used in the second polymer material may be configured to strongly bond with the thermoset polymer in the first polymer material coating. The thermoplastic polymer may also bond with the thermoset polymers, but the thermoplastic polymer may have different properties than the thermoset polymers, including at least a different melting point and flowability in response to increased temperature and melting, as further described elsewhere herein. The thermoplastic polymer may comprise any of the other thermoplastic polymers described elsewhere herein.

In some embodiments, the second polymer material may be applied in layers to the first polymer material. Each layer of the second polymer material may, for example, begin to (or completely) cure before each additional layer of the second polymer material is applied to the tubular shape. Over time, the second polymer material may be applied with a specified thickness. The overall thickness of the second polymer material layers in a completed catheter may be within a range of between about 5 percent of the thickness of the base/first polymer material layers to about 200 percent of the thickness of the base/first polymer material layers. The relative thicknesses of the first and second polymer materials may help define the overall flowability of the completed catheter, wherein lower relative amounts of the second polymer material may make the outer surface of the catheter less flowable and meltable than catheters with higher proportions of the second polymer material relative to the first polymer material.

Block 608 indicates that the first and second polymer materials may be cured. The curing step may be completed in stages, such as by curing one or more layers of polymer material between applications of the polymer materials to the mandrel or to other layers of polymer material. For example, the first polymer material coating may be completely cured before applying the second polymer material. In other embodiments, all of the coatings may be cured simultaneously. Thus, curing the first and second polymer materials may comprise curing each of the polymer materials separately or curing them simultaneously. In some embodiments, the polymer materials may thermally cure, and in other embodiments, the polymer materials may cure as a result of exposure of the polymer materials to a catalyst (e.g., ultraviolet radiation).

Block 610 indicates that the mandrel may be removed from within the first and second polymer materials. Removal of the mandrel may leave behind a tubular member having layers of the first polymer material and the second polymer material, similar to the tube 202 disclosed above.

In some arrangements, the first polymer material may comprise the thermoset and thermoplastic polymer materials, and the second polymer material may comprise the thermoset polymer material. In this instance, the inner surface of the catheter may be configured to melt or soften upon exposure to heat, and the inner surface may be a strike layer used to bond the catheter to another cylindrical member.

In some embodiments, the method 600 may be used to manufacture a tube having a plurality of layers (e.g., catheter 400). Thus, the method 600 may further comprise applying one or more intermediate layers between the innermost layer of the first polymer material and the outermost layer of the second polymer material. The intermediate layers may comprise a mixed and blended composition of thermoset polymer material and thermoplastic polymer material, wherein the intermediate layers may comprise a higher concentration of thermoset polymer material relative to the thermoplastic polymer material than the outer layers of the second polymer material.

Similarly, the method 600 may be used to manufacture a tube having a substantially continuous transition of materials from its innermost surface to its outermost surface (e.g., catheter 500). In this configuration, the layers of polymer material applied in separate coats to the mandrel may each slightly differ in composition such that, after applying many thin layers, the layers have a progressively higher and higher concentration of thermoplastic polymer material (relative to thermoset polymer material in each layer) the further they are from the central axis of the mandrel. After applying many layers, a catheter (e.g., 500) with a continuous, gradual concentration change may be constructed and removed from the mandrel. The outer-most boundary of the tube (i.e., the highest radius from the center of the mandrel) may have a higher concentration of thermoplastic polymer material than the inner-most boundary of the tube (e.g., the surface of the tube that contacts or is immediately adjacent to the surface of the mandrel).

In some arrangements, the method 600 may further include providing a second tubular member, with the second tubular member having an inner surface. The method may also include approximating the inner surface of the second tubular member and the second polymer material, heating the second polymer material, thereby at least partially melting the thermoplastic polymer, and bonding the second polymer material to the inner surface of the second tubular member. In this manner, the manufactured tube may be connected and bonded to the second tubular member without the use of externally-applied adhesives. In these additional steps, an attachment mandrel may be used that extends through a lumen of the second tubular member and the tubular member made from the first and second polymer materials.

Various inventions have been described herein with reference to certain specific embodiments and examples. However, they will be recognized by those skilled in the art that many variations are possible without departing from the scope and spirit of the inventions disclosed herein, in that those inventions set forth in the claims below are intended to cover all variations and modifications of the inventions disclosed without departing from the spirit of the inventions. The terms “including:” and “having” come as used in the specification and claims shall have the same meaning as the term “comprising.”

What is claimed is:

1. A tube having a bonding surface, the tube comprising:
a tubular body having a central lumen, the tubular body comprising:
a longitudinal axis;
a tubular thickness, the tubular thickness extending through the tubular body in a radial direction relative to the longitudinal axis;
a first thickness layer, the first thickness layer comprising a thermoset polymer material;
a second thickness layer, the second thickness layer comprising a thermoplastic polymer material, the thermoplastic polymer material comprising at least one of polyether block amide, polyurethane, nylon, or polycarbonate.

2. The tube of claim 1, wherein the second thickness layer comprises the thermoset polymer material and the thermoplastic polymer material.

3. The tube of claim 1, wherein the thermoset polymer material comprises polyimide and the thermoplastic polymer material comprises polyether block amide.

4. The tube of claim 1, wherein the thermoplastic polymer material is bondable to the thermoset polymer material upon heating the second thickness layer.

5. The tube of claim 1, wherein the first thickness layer is positioned radially internal to the second thickness layer.

6. The tube of claim 1, wherein the first thickness layer is positioned radially external to the second thickness layer.

7. The tube of claim 1, wherein the first and second thickness layers are bonded to each other.

8. The tube of claim 1, further comprising a third thickness layer, the third thickness layer being positioned radially between the first and second thickness layers, the third thickness layer comprising the thermoset polymer material and the thermoplastic polymer material.

9. The tube of claim 8, wherein the thermoset polymer material and the thermoplastic polymer material are blended in the third thickness layer.

10. A tubular catheter apparatus, comprising:
a tubular catheter, the tubular catheter comprising a first longitudinal axis and a first surface, the first surface extending circumferentially around the first longitudinal axis;
a catheter tip, the catheter tip comprising a second longitudinal axis, a second surface, and a third surface, the second surface extending circumferentially around the second longitudinal axis, the third surface extending circumferentially around the second longitudinal axis and being radially spaced from the second surface, the second surface comprising a thermoset polymer material, the third surface comprising a thermoplastic polymer material, the thermoplastic polymer material being bonded to the first surface, the thermoplastic polymer material comprising at least one of polyether block amide, polyurethane, nylon, or polycarbonate;
wherein the tubular catheter and the catheter tip are in fluid communication with each other.

11. The tubular catheter apparatus of claim 10, wherein the first surface is positioned radially external to the third surface.

12. The tubular catheter apparatus of claim 11, wherein the catheter tip comprises a first end portion comprising the second and third surfaces and a second end portion positioned opposite the first end portion, the second end portion having a greater outer diameter than the first end portion.

13. The tubular catheter apparatus of claim 10, wherein the tubular catheter and the catheter tip each have constant outer diameters along the first and second longitudinal axes.

14. The tubular catheter apparatus of claim 10, wherein the first surface is positioned radially internal to the third surface.

15. A method of bonding surfaces of polymer materials, the method comprising:
providing a first bonding surface and a second bonding surface, the first bonding surface comprising a thermoset polymer material, the second bonding surface comprising the thermoset polymer material and a thermoplastic polymer material, the thermoplastic polymer material comprising at least one of polyether block amide, polyurethane, nylon, or polycarbonate;
applying heat to the second bonding surface;
bringing the first and second bonding surfaces into contact with each other;
cooling the first and second bonding surfaces, wherein the thermoplastic polymer material is bonded with the first bonding surface.

16. The method of claim 15, wherein the first bonding surface is positioned on a first device and the second bonding surface is positioned on a second device, the second device comprising a first layer and a second layer, the first layer comprising the second bonding surface, the second layer comprising the thermoset polymer material, the first layer being bonded to the second layer.

17. The method of claim 16, wherein the first and second devices are tubular.

18. The method of claim 15, wherein applying heat to the second bonding surface at least partially melts the thermoplastic polymer material at the second bonding surface.

19. The method of claim 15, wherein the thermoset polymer material is polyimide.

20. The method of claim 15, wherein the thermoset polymer material and the thermoplastic polymer material are blended together in the second bonding surface.

21. A method of forming a tubular catheter, the method comprising:
providing a mandrel;
coating the mandrel with a first polymer material, the first polymer material comprising a thermoset polymer, the thermoset polymer forming a tubular shape around the mandrel;
coating the tubular shape with a second polymer material, the second polymer material comprising the thermoset polymer and a thermoplastic polymer, the thermoplastic polymer comprising at least one of polyether block amide, polyurethane, nylon, or polycarbonate;
curing the first and second polymer materials;
removing the mandrel from the first and second polymer materials.

22. The method of claim 21, wherein the thermoset polymer and the thermoplastic polymer are blended in the second polymer material.

23. The method of claim 21, wherein the second polymer material comprises an inner boundary and an outer boundary, the outer boundary having a higher concentration of the thermoplastic polymer than the inner boundary.

24. The method of claim 21, further comprising:
providing a second tubular member, the second tubular member having an inner surface;
approximating the inner surface of the second tubular member and the second polymer material;
heating the second polymer material, thereby at least partially melting the thermoplastic polymer;
bonding the second polymer material to the inner surface of the second tubular member.

25. The method of claim 24, wherein the step of approximating the inner surface of the second tubular member and the second polymer material comprises positioning an attachment mandrel within the first polymer material, and wherein the attachment mandrel remains within the first polymer material until the second polymer material is bonded to the inner surface of the second tubular member.
